(19) United States
(12) Patent Application Publication
(10) Pub. No.: US 2025/0215182 A1
(43) Pub. Date: Jul. 3, 2025

(54) CIRCULAR ECONOMY FOR WASTE POLYSTYRENE VIA REFINERY FCC UNIT

(71) Applicant: Chevron U.S.A. Inc., San Ramon, CA (US)

(72) Inventors: Joel E. SCHMIDT, San Ramon, CA (US); Hye-Kyung Cho TIMKEN, Albany, CA (US); Tengfei LIU, San Ramon, CA (US)

(73) Assignee: Chevron U.S.A. Inc., San Ramon, CA (US)

(21) Appl. No.: 18/999,141

(22) Filed: Dec. 23, 2024

Related U.S. Application Data
(60) Provisional application No. 63/615,373, filed on Dec. 28, 2023.

(51) Int. Cl. C08J 11/20; C07C 4/22

(52) U.S. Cl. CPC C08J 11/20; C07C 4/22; C07C 2523/02; C08J 2325/06

(57) ABSTRACT

Provided is a process for converting waste polystyrene plastic into recycle for polystyrene polymerization. The process comprises selecting waste polystyrene plastics and preparing a stable blend of aromatic-rich hydrocarbon feedstock and the polystyrene plastic. The amount of plastic in the blend comprises no more than 20 wt. % of the blend. The blend is passed to a refinery FCC unit. Aromatic products are recovered from the FCC unit, from which styrene is separated. The separated styrene can then be passed to a polymerization unit to prepare polystyrene.

### CROSS REFERENCE TO RELATED APPLICATIONS

The present application claims priority to U.S. Provisional Application No. 63/615,373 filed Dec. 28, 2023, the complete disclosure of which is incorporated herein by reference in its entirety.

### BACKGROUND

The world has seen extremely rapid growth of plastics production. According to PlasticEurope Market Research Group, the world's plastics production was 335 million tons in 2016, 348 million tons in 2017 and 359 million tons in 2018. According to McKinsey & Company, the global plastics-waste volume was estimated about 260 million tons per year in 2016 and projected to be 460 million tons per year by 2030 if the current trajectory continues.

Single use plastic waste has become an increasingly important environmental issue. At the moment, there appear to be few options for recycling polyethylene and polypropylene waste plastics to value-added chemicals and fuel products. Currently, only a small amount of polyethylene/polypropylene is recycled via chemical recycling, where recycled and cleaned plastic pellets are pyrolyzed in a pyrolysis unit to make fuels (naphtha, diesel), steam cracker feed or slack wax. The majority, greater than 80%, is incinerated, land filled or discarded.

The current method of the chemical recycling via pyrolysis cannot make a big impact for the plastics industry. The current pyrolysis operation produces poor quality fuel components (naphtha and diesel range products), but the quantity is small enough that these products can be blended into fuel supplies. However, this simple blending cannot continue if we have to recycle very large volumes of waste polyethylene and polypropylene to address the environmental issues. The products as produced from the pyrolysis unit have too poor quality to be blended in large amounts (for example 5-20 volume % blending) in transportation fuels.

Processes are known which convert waste plastic into hydrocarbon lubricants. For example, U.S. Pat. No. 3,845,157 discloses cracking of waste or virgin polyolefins to form gaseous products such as ethylene/olefin copolymers which are further processed to produce synthetic hydrocarbon lubricants. U.S. Pat. No. 4,642,401 discloses the production of liquid hydrocarbons by heating pulverized polyolefin waste at temperatures of 150-500° C. and pressures of 20-300 bars. U.S. Pat. No. 5,849,964 discloses a process in which waste plastic materials are depolymerized into a volatile phase and a liquid phase. The volatile phase is separated into a gaseous phase and a condensate. The liquid phase, the condensate and the gaseous phase are refined into liquid fuel components using standard refining techniques. U.S. Pat. No. 6,143,940 discloses a procedure for converting waste plastics into heavy wax compositions. U.S. Pat. No. 6,150,577 discloses a process of converting waste plastics into lubricating oils. EP0620264 discloses a process for producing lubricating oils from waste or virgin polyolefins by thermally cracking the waste in a fluidized bed to form a waxy product, optionally using a hydrotreatment, then catalytically isomerizing and fractionating to recover a lubricating oil.

U.S. Pub. No. 2021/0130699 discloses processes and systems for making recycle content hydrocarbons from recycled waste material. The recycle waste material is pyrolyzed to form a pyrolysis oil composition, at least a portion of which may then be cracked to form a recycle olefin composition.

Other documents which relate to processes for converting waste plastic into lubricating oils include U.S. Pat. Nos. 6,288,296; 6,774,272; 6,822,126; 7,834,226; 8,088,961; 8,404,912 and 8,696,994; and U.S. Patent Application Publication Nos. 2019/0161683; 2016/0362609; and 2016/0264885. The foregoing patent documents are incorporated herein by reference in their entirety.

Globally, recycling or upcycling of plastic waste has gained great interest to save resources and the environment. Mechanical recycling of plastic waste is rather limited due to different types, properties, additives, and contaminants in the collected plastics. Usually, the recycled plastics are of degraded quality. Chemical recycling to the starting material or value-added chemicals has emerged as a more desirous route.

While plastics such as polyethylene and polypropylene have been the focus of chemical recycling, there are many other waste plastics. Such plastics have been ignored to some extent due to difficulties in chemical recycling. One such waste plastic is polystyrene.

Polystyrene is a high use plastic that finds uses ranging from packaging materials and foams to hard containers. Currently there is little incentive to recycle polystyrene. As the main constituent of polystyrene is an aromatic species it may be beneficial if this can be recovered as styrene to then make circular polystyrene or as another aromatic species that may have higher value than typical plastic pyrolysis products. Indeed, it has been reported that styrene and other aromatic species can be recovered from the pyrolysis of polystyrene.

Pyrolysis of solid plastics is an important technology as it offers a method for dealing with hard to recycle plastics such as polyethylene, polypropylene, and polystyrene. However thermal pyrolysis has some serious downsides such as the formation of high amounts of dry gases, which are used to fuel the pyrolysis process but have no other value, and solid char that needs to be landfilled. Catalytic pyrolysis is a known process which can improve yields of desired products but practicing this process with a solid feed is challenging.

However, in order to achieve chemical recycling of polystyrene plastics in an industrially significant quantity to reduce its environmental impact, more robust processes are needed. Such a process may require unique handing and manipulation of the polystyrene waste plastic. The improved process should also establish a “circular economy” for the waste polystyrene where the spent polystyrene is effectively recycled as starting materials for polymers or value-added chemicals and fuels, including gasoline.

### SUMMARY

Provided is a continuous process for converting waste polystyrene into recycle for polystyrene polymerization. The process comprises selecting waste polystyrene plastics and blending the polystyrene with an aromatic-rich hydrocarbon feedstock. The resulting blend is generally a stable blend and a uniform physical mixture, particularly at a temperature below the melting point of the polystyrene plastic. The blend comprises about 20 wt. % or less of the polystyrene waste plastic. The blend is then cofed with conventional refinery feed, such as VGO, to a FCC unit in a refinery.

The incorporation of the process with an oil refinery is an important aspect of the present process and allows the creation of a circular economy with a single use waste plastic such as polystyrene. Thus, the blend is passed to a refinery FCC unit. The blend is passed at a temperature above its pour point in order to be able to pump the blend to the refinery FCC unit. The blend is heated above the melting point of the polystyrene plastic before being injected to the reactor. By selecting an appropriate catalyst for the FCC unit, a styrene and aromatics mixture is recovered from the FCC unit. In one embodiment, a metal oxide catalyst, such as a calcium and/or magnesium oxide containing additive catalyst, is used in the FCC unit. The mixture can be treated to remove styrene from the mixture. The styrene can then be passed to a polymerization unit to prepare polystyrene.

The refinery will generally have its own hydrocarbon feed flowing through the refinery units. An important aspect of the present process is to not negatively impact the operation of the refinery. The refinery must still produce valued chemicals and fuels. Otherwise, the incorporation of the process with an oil refinery would not be a workable solution. The flow volume must therefore be carefully observed.

The flow volume of the waste plastic/hydrocarbon blend to the refinery units can comprise any practical or accommodating volume % of the total flow to the refinery units. Generally, the flow of the blend can be up to about 100 vol. % of the total flow, i.e., the blend flow is the entire flow, with no refinery flow. In one embodiment, the flow of the blend is an amount up to about 50 vol. % of the total flow, i.e., the refinery flow and the blend flow.

Among other factors, it has been found that by adding refinery operations one can efficiently and effectively recycle plastic waste while also complementing the operation of a refinery in the preparation of higher value products such as gasoline, jet fuel, base oil and diesel. But also, by adding refinery operations and selecting appropriate catalysts for the FCC unit, it has been found that clean styrene can be efficiently and effectively produced from the waste polystyrene plastics for ultimate polystyrene polymer production. Positive economics are realized for the overall process from recycled plastics to a polystyrene product with product quality identical to that of virgin polymer.

### BRIEF DESCRIPTION OF THE DRAWINGS

FIG. 1 depicts the current practice of pyrolyzing waste plastics to produce fuel or wax (base case).

FIG. 2 depicts a present process of preparing a hot, homogenous liquid blend of plastic and hydrocarbon feedstock, and how the blend can be fed to a refinery conversion unit.

FIG. 3 depicts in detail a stable blend preparation process, and how the stable blend can be fed to a refinery conversion unit.

FIG. 4 depicts a present process where the prepared blend is passed to a refinery FCC unit.

FIG. 5 depicts a present process for establishing a circular economy for waste plastics where the blend is passed to a refinery FCC feed pre-treater before a refinery FCC unit.

FIG. 6 graphically shows the thermal stability of various plastics, including polystyrene, by Thermal Gravimetric Analysis (TGA) results.

### DETAILED DESCRIPTION

In the present process, provided is a method to recycle waste polystyrene back to virgin polystyrene to establish a circular economy by combining distinct industrial processes. A substantial portion of polystyrene polymers are used in single use plastics and get discarded after its use. The single use plastic waste has become an increasingly important environmental issue. At the moment, there appear to be few options for recycling polystyrene waste plastics to value-added chemicals and fuel products. Currently, only a small amount if any, polystyrene is recycled via chemical recycling, using pyrolysis.

The present process, however, does not pyrolyze the waste polystyrene plastic. Rather, a stable blend of aromatic-rich hydrocarbon feedstock and the waste polystyrene plastic is prepared. Thus, the pyrolysis step can be avoided, which is a significant energy savings.

The stable blend is made by a two-step process. The first step produces a hot, homogeneous liquid blend of polystyrene plastic melt and hydrocarbon feedstock. The preferred range of the plastic composition in the blend is about 1-20 wt. %, 1-10 wt. % in one embodiment, and from 1-5 wt. % in another embodiment. The preferred conditions for the hot liquid blend preparation include heating of polystyrene plastic above the melting point of the plastic while vigorously mixing with the hydrocarbon feedstock, which can comprise LCO, heavy gasoline, heavy reformate and/or an aromatic solvent. The preferred process conditions include heating to a 250-550° F. (150-290° C.) temperature with a residence time of 5-240 minutes at the final heating temperature, and 0-10 psig atmospheric pressure. This can be done in the open atmosphere as well as preferably under an oxygen-free inert atmosphere.

In the second step, the hot blend is cooled down below the melting point of the plastic while continuously, vigorously mixing, and then further cooling down to a lower temperature, preferably an ambient temperature, to produce a stable blend. The stable blend is either an oily liquid or in a waxy solid state at the ambient temperature depending on the hydrocarbon feedstock.

In one embodiment, the stable blend is made of the hydrocarbon feedstock and 1-20 wt. % of waste polystyrene plastic, wherein the polystyrene plastic is in the form of finely dispersed micron-size particles with 10 micron to less than 100-microns average particle size.

There are several advantages realized by the present blend and its use. For example, the stable blend of polystyrene plastic and hydrocarbon feedstock can be stored at ambient temperature and pressure for extended time periods. During the storage, no agglomeration, no settling of polymer particles and no chemical/physical degradation of the blend are observed. This allows easier handling of the waste plastic material for storage or transportation.

The stable blend can be handled easily by using standard pumps as are typically used in refineries or warehouses, or by using pumps equipped with transportation tanks. Depending on the blend, some heating of the blend above its pour point is required to pump the blend for transfer or for feeding to a conversion unit in a refinery. During the heating, no agglomeration of polystyrene polymer is observed.

For feeding to a refinery conversion unit, the stable blend can be further heated above the melting point of the polystyrene plastic to produce a homogeneous liquid blend of hydrocarbon and polystyrene plastic. The hot homogeneous liquid blend is fed directly to the oil refinery process units for conversion of waste polystyrene plastics to high value products with good yields.

In one embodiment, the blend is prepared in a hot blend preparation unit where the operating temperature is above the melting point of the plastic (about 150-290° C.), to make a hot, homogeneous liquid blend of plastic and oil. The hot homogeneous liquid blend of plastic and oil can be fed directly to the refinery units.

Alternatively, a blend is prepared in a stable blend preparation unit where the hot homogeneous liquid blend is cooled to ambient temperature in a controlled manner to allow for easy storage and transportation. By using this method, a stable blend can be prepared at a facility away from a refinery and can be transported to a refinery unit. Then the stable blend is heated above the melting point of the plastic to feed to the refinery conversion unit. The stable blend is a physical mixture of microcrystalline plastic particles finely suspended in the hydrocarbon feedstock. The mixture is stable, and the plastic particles do not settle or agglomerate upon storage for extended period.

What is meant by heating the blend to a temperature above the melting point of the plastic is clear when a single plastic is used. However, if the waste plastic comprises more than one waste plastic, then the melting point of the plastic with the highest melting point is exceeded. Thus, the melting points of all plastics must be exceeded. Similarly, if the blend is cooled below the melting point of the plastic, the temperature must be cooled below the melting points of all plastics comprising the blend.

Compared with the pyrolysis unit, these blend preparation units operate at a much lower temperature (˜500-600° C. vs. 120-290° C.). Thus, the present process is a far more energy efficient process in preparing a refinery feedstock derived from waste plastic than a thermal cracking process such as pyrolysis.

The use of the present waste plastic/hydrocarbon blend further increases the overall hydrocarbon yield obtained from the waste plastic. This increase in yield is significant. The hydrocarbon yield using the present blend offers a hydrocarbon yield that can be as much as 98%. To the contrary, pyrolysis produces a significant amount of light product from the plastic waste, about 10-30 wt. %, and about 5-10 wt. % of char. These light hydrocarbons are used as fuel to operate the pyrolysis plant, as mentioned above. Thus, the liquid hydrocarbon yield from the pyrolysis plant is at most 70-80%.

When the present blend is passed into the refinery units, such as a FCC unit, only a minor amount of offgas is produced. Refinery units use catalytic cracking processes that are different from the thermal cracking process used in pyrolysis. With catalytic processes, the production of undesirable light-end byproducts such as methane and ethane is minimized. Refinery units have efficient product fractionation and are able to utilize all hydrocarbon products streams efficiently to produce high value materials. Refinery co-feeding will produce only about 2% of offgas (H₂, methane, ethane, ethylene). The C₃ and C₄ streams are captured to produce useful products such as circular polymer and/or quality fuel products. Thus, the use of the present hydrocarbon/plastic blend offers increased hydrocarbons from the plastic waste, as well as a more energy efficient recycling process compared to a thermal process such as pyrolysis.

The present process converts single use waste polystyrene plastic in large quantities by integrating the waste plastic blended with hydrocarbon product streams into an oil refinery operation. The resulting processes can produce aromatics, including styrene. Styrene can be recovered in more significant amounts should an appropriate selection of catalyst for the conversion unit be made. Metal oxide containing additive catalysts, such as calcium and magnesium oxide additive catalysts, result in more significant amounts of styrene. The styrene can be used to prepare polystyrene.

Generally, the present process provides a circular economy for polystyrene plants. Polystyrene is produced via polymerization of pure styrene. The styrene can be separated from the products produced by cracking the blend in an FCC unit, provided appropriate catalysts are selected for the FCC unit.

By adding refinery operations to upgrade the waste plastic to higher value products (gasoline, jet fuel and diesel, base oil) and to produce clean styrene for ultimate polystyrene polymer production, positive economics are realized for the overall process of recycled plastics to polystyrene products with product quality identical to that of the virgin polymer. And, by integrating the present recycle process with an oil refinery operation, a more energy efficient and effective process is achieved while avoiding any issues with the refinery operation.

The integration of a refinery operation becomes quite important in another aspect. Waste plastics contain contaminants, such as calcium, magnesium, chlorides, nitrogen, sulfur, dienes, and heavy components, and these products cannot be used in a large quantity for blending in transportation fuels. It has been discovered that by having these products go through the refinery units, the contaminants can be captured in pre-treating units and their negative impacts diminished. The fuel components can be further upgraded with appropriate refinery units using chemical conversion processes, with the final transportation fuels produced in the integrated process being of higher quality and meeting the fuels quality requirements. The integrated process will generate a cleaner and more pure styrene stream for polystyrene production. These large on-spec productions allow “cyclical economy” for the recycled plastics to be feasible.

The carbon in and out of the refinery operations are “transparent,” meaning that all the molecules from the waste plastic do not necessarily end up in the exact styrene product cycled back to the polystyrene plants, but are nevertheless assumed as “credit” as the net “green” carbon in and out of the refinery is positive. With these integrated processes, the amount of virgin feeds needed for polystyrene plants are reduced significantly.

The stable blend of polystyrene plastic and hydrocarbon feedstock allows more efficient recycling of waste polystyrene plastics. The use of the present blend is far more energy efficient than the current pyrolysis process, and allows recycling with a lower carbon footprint. The improved processes would allow establishment of a circular economy on a much larger scale by efficiently converting waste polystyrene plastics back to virgin quality polymers or value-added chemicals and fuels.

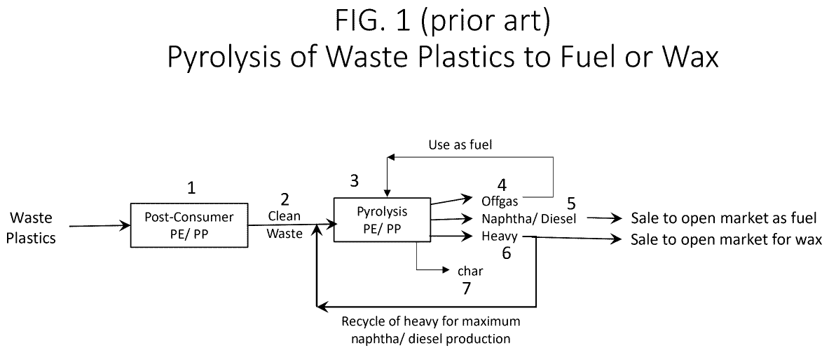

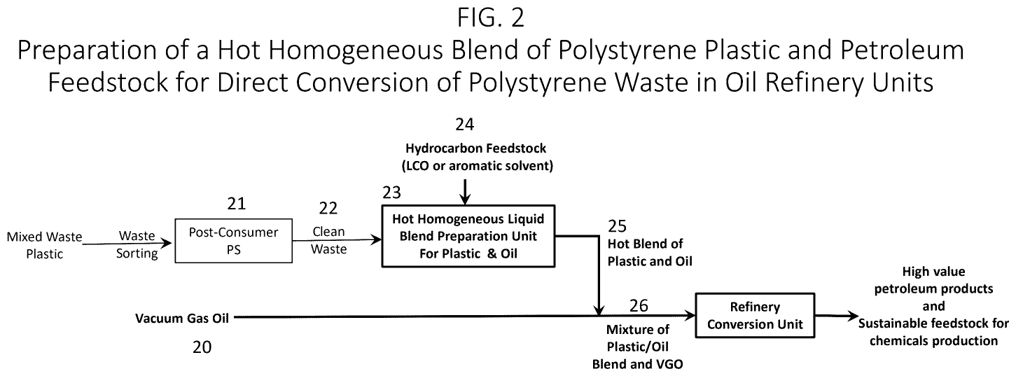

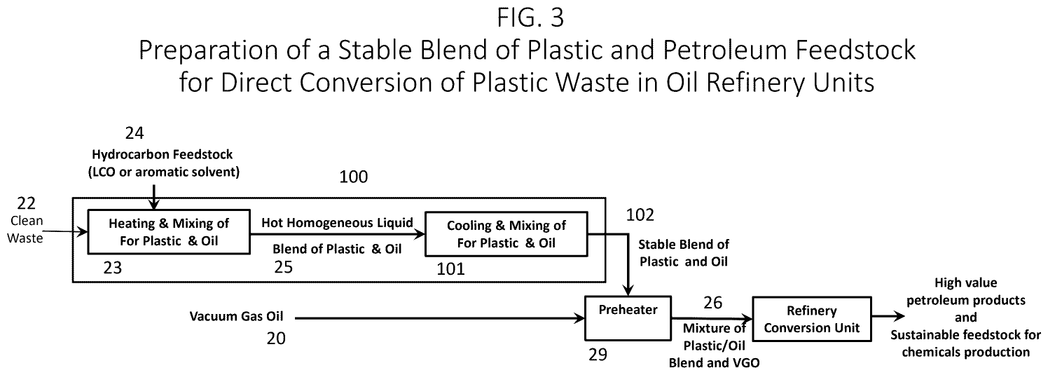

A simplified process diagram for a base case of a waste plastics pyrolysis process is shown in FIG. 1. Preparation of a hot homogeneous liquid blend of polystyrene plastic and hydrocarbon feedstock is shown in FIG. 2. FIG. 3 depicts in detail the preparation of a stable blend of waste plastic and hydrocarbon based feedstock. The figures depict the two process steps associated with the blend preparation.

As noted above, FIG. 1 shows a diagram of the pyrolysis of waste plastics fuel or wax that is generally operated in the industry today. Generally, the waste plastics are sorted together 1. The cleaned plastic waste 2 is converted in a pyrolysis unit 3 to offgas 4 and pyrolysis oil (liquid product). The offgas 4 from the pyrolysis unit 3 is used as fuel to operate the pyrolysis unit. An on-site distillation unit separates the pyrolysis oil to produce naphtha and diesel 5 products which are sold to fuel markets. The heavy pyrolysis oil fraction 6 is recycled back to the pyrolysis unit 3 to maximize the fuel yield. Char 7 is removed from the pyrolysis unit 3. The heavy fraction 6 is rich in long chain, linear hydrocarbons, and is very waxy (i.e., forms paraffinic wax upon cooling to ambient temperature). Wax can be separated from the heavy fraction 6 and sold to the wax markets.

Use of the present blend, however, avoids the pyrolysis of the waste plastic. Rather, a stable blend of hydrocarbon feedstock and the polystyrene plastic is prepared, which can be fed to the refinery units. Thus, the pyrolysis step can be avoided, which is a significant energy savings.

FIG. 2 illustrates a method for preparing a hot homogenous blend of polystyrene plastic and hydrocarbon feedstock which can be used for direct injection to a refinery unit. The preferred range of the plastic composition in the blend is about 1-20 wt. %, but can range from 1-10 wt. % in one embodiment, or 1-5 wt. % in another embodiment. If high molecular weight polystyrene (average molecular weight of 150,000 to 500,000 or greater) waste plastic is used as the predominant waste plastic, e.g., at least 50 wt. %, then the amount of waste plastic used in the blend is more preferably about 10 wt. %. The reason being that the pour point and viscosity of the blend would be high. In one embodiment, the plastic can comprise polystyrene having an average molecular weight, M_{w}, in the range of 5,000 to 50,000. In another embodiment, the polystyrene plastic can comprise polystyrene having an average molecular weight, M_{w}, in the range of 50,000 to 250,000.

The preferred conditions for the hot homogeneous liquid blend preparation include heating the plastic above the melting point of the plastic while vigorously mixing with a hydrocarbon feedstock. The preferred process conditions include heating to a 250-550° F. (120-290° C.) temperature, although always less than 550° F., with a residence time of 5-240 minutes at the final heating temperature, and 0-10 psig atmospheric pressure. This can be done in an open atmosphere as well as under an oxygen-free inert atmosphere.

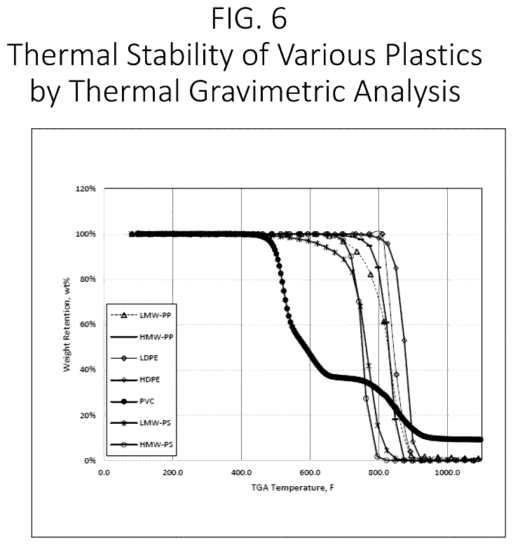

The hot homogeneous blend of polystyrene plastic melt and hydrocarbon feedstock is prepared by mixing a hydrocarbon feed and a polystyrene plastic together and then heating the mixture above the melting point of the polystyrene plastic, but not greater than 550° F. (290° C.), while thoroughly mixing. The heating temperature should not be so high as to begin breakdown of polystyrene plastic. FIG. 6 graphically shows Thermal Gravimetric Analyses (TGA) results for various plastics, including polystyrene. The results indicate the thermal stabilities of the various plastics. Alternatively, the blend is prepared by melting the polystyrene plastic only and then adding the polystyrene plastic melt to the warm or hot hydrocarbon feedstock while thoroughly mixing. Alternatively, it is prepared by heating the hydrocarbon only to the temperature above the melting point of the polystyrene plastic and then adding solid polystyrene plastic slowly to the hot hydrocarbon liquid while thoroughly mixing the mixture and maintaining the temperature above the melting point of the plastic.

Referring to FIG. 2 of the Drawings, a stepwise preparation process of preparing the hot homogeneous liquid blend is shown. Mixed waste plastic is sorted to create post-consumer waste plastic 21 comprising polyethylene and/or polypropylene. The waste plastic is cleaned 22 and then mixed with an oil 24 in a hot blend preparation unit 23. After the mixing in 23, the homogeneous blend of the plastic and oil is recovered 25. Optionally a filtration device may be added (not shown) to remove any undissolved plastic particles or any solid impurities present in the hot liquid blend. The hot blend of the plastic and oil is then combined with the refinery feedstock, such as vacuum gas oil (VGO) 20, and becomes a mixture of the plastic/oil blend and VGO, 26, which can then be passed to a refinery unit.

FIG. 3 illustrates a method for preparing a stable blend of plastic and oil. The stable blend is made in a stable blend preparation unit by a two-step process. The first step produces a hot, homogeneous liquid blend of polystyrene plastic melt and hydrocarbon feedstock, the step is identical to the hot blend preparation described in FIG. 2. The preferred range of the plastic composition in the blend is about 1-20 wt. %. If high molecular weight polystyrene waste plastic is used as the predominant waste plastic, e.g., at least 50 wt. %, then the amount of waste plastic used in the blend is more preferably about 10 wt. %. The reason being that the pour point and viscosity of the blend would be high.

The preferred conditions for the hot homogeneous liquid blend preparation include heating the plastic above the melting point of the plastic while vigorously mixing with a hydrocarbon feedstock. The preferred process conditions include heating to a 250-550° F. (120-290° C.) temperature, with a residence time of 5-240 minutes at the final heating temperature, and 0-10 psig atmospheric pressure. This can be done in an open atmosphere as well as under an oxygen-free inert atmosphere.

In the second step, the hot blend is cooled down below the melting point of the plastic while continuously vigorously mixing, and cooling to a lower temperature, preferably ambient temperature, to produce a stable blend of the plastic and oil.

It has been found that the stable blend is an intimate physical mixture of polystyrene plastic and hydrocarbon feedstock. The polystyrene plastic is in a “de-agglomerated” state. The polystyrene plastic maintains a finely dispersed state of solid particles in the hydrocarbon feedstock at temperatures below the melting point of the plastic, and particularly at ambient temperatures. The blend is stable and allows easy storage and transportation. At a refinery, the stable blend can be heated in a preheater above the melting point of the plastic to produce a hot, homogenous liquid blend of the plastic and hydrocarbon. The hot liquid blend can then be fed to a refinery unit as a cofeed with conventional refinery feed.

In FIG. 3, further details of the stable blend preparation are shown. The stable blend is made in a stable blend preparation unit 100 by a two-step process. As shown, clean polystyrene waste 22 is passed to the stable blend preparation unit 100. The selected plastic waste 22 is heated and mixed with a hydrocarbon feedstock oil 24. The plastic waste is heated above the melting point of the plastic to melt the plastic. The hydrocarbon feedstock is mixed with the heated plastic at 23. The mixing is often quite vigorous. The mixing and heating conditions can generally comprise heating at a temperature in the range of about 250-550° F. (120-290° C.), with a residence time of 5-240 minutes at the final heating temperature. The heating and mixing can be done in the open atmosphere or under an oxygen-free inert atmosphere. The result is a hot, homogenous liquid blend of plastic and oil 25. Optionally a filtration device may be added (not shown) to remove any undissolved plastic particles or any solid impurities present in the hot homogeneous liquid blend.

The hot blend 25 is then cooled below the melting point of the plastic while continuing the mixing of the plastic with the hydrocarbon oil feedstock at 101. Cooling generally continues, usually to an ambient temperature, to produce a stable blend of the plastic and oil 102. At a refinery, the stable blend can be fed to a preheater, 29, which heats the blend above the melting point of the plastic to produce a mixture of plastic/oil blend and VGO, 26, which is then fed to a refinery conversion unit.

The present plastic starting material for use in the present blend comprises polystyrene. The pre-sorted polystyrene is washed and shredded or pelleted to feed to a blend preparation unit. Washing of the polystyrene can remove metal contaminants such as sodium, calcium, magnesium, aluminum, and non-metal contaminants coming from other waste sources. Non-metal contaminants include contaminants coming from the Periodic Table Group IV, such as silica, contaminants from Group V, such as phosphorus and nitrogen compounds, contaminants from Group VI, such as sulfur compounds, and halide contaminants from Group VII, such as fluoride, chloride, and iodide. The residual metals, non-metal contaminants, and halides need to be removed to less than 50 ppm, preferentially less than 30 ppm and most preferentially to less than 5 ppm.

The hydrocarbon with which the polystyrene is blended is generally rich in aromatics. In one embodiment, the hydrocarbon feedstock oil with which the waste polystyrene plastic is blended comprises light cycle oil (LCO), medium cycle oil (MCO), heavy cycle oil (HCO), FCC naphtha, gasoline, and/or an aromatic solvent derived from conventional petroleum refining. In one embodiment, the aromatic-rich hydrocarbon feedstock is derived from biofeedstock processing where the oxygen has been removed and the remaining liquid products are mostly aromatic hydrocarbons. It has been found that blending polystyrene with light cycle oil and/or an aromatic solvent derived from petroleum provides a very stale blend, and is thus preferred. Only an oil with high aromatic content can make a stable blend with polystyrene. Paraffinic feedstocks such as hydrotreated vacuum gas oil, paraffinic solvent, bio feedstock, do not make a stable blend with polystyrene.

The aromatics in the aromatic-rich hydrocarbon feedstock can range from 50 wt. % to 99 wt. % of the hydrocarbon feedstock, and generally comprises 1-ring, 2-ring and 3-ring aromatics. More preferably, the aromatic-rich hydrocarbon feedstock comprises 75 wt. % and higher of 1-ring, 2-ring, and 3-ring aromatics.

More than one hydrocarbon feedstock can be used to optimize the blend properties. For example, the viscosity and pour point can be adjusted by adding different hydrocarbon feedstocks.

Optionally, solvents such as benzene, toluene, or xylene may be added to the blend to reduce the viscosity or pour point of the blend of polystyrene plastic and hydrocarbon feedstock for easier handling.

While not wanting to be bound by a theory, the present process prepares a stable blend that is an intimate physical mixture of plastic and hydrocarbon feedstock for catalytic conversion in refinery units. The present process produces a stable blend of hydrocarbon feedstock and plastic wherein the plastic is in a “de-agglomerated” state. The polystyrene plastic maintains its state as “finely dispersed” solid particles in the hydrocarbon feedstock at ambient temperature. This blend is stable and allows easy storage and transportation. At a refinery, the stable blend can be preheated above the melting point of the plastic to produce a hot, homogeneous liquid blend of plastic and hydrocarbon, and then the hot liquid blend is fed to a conversion unit. Then both the hydrocarbon feed and plastic are simultaneously converted in the conversion unit with typical refinery catalysts containing zeolite(s) and other active components such as silica-alumina, alumina and clay.

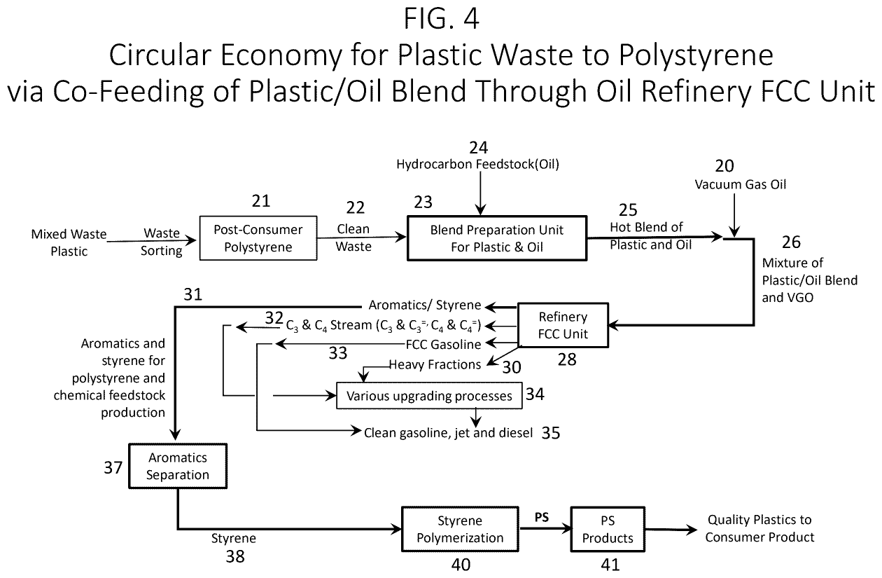

FIG. 4 shows one embodiment of a present integrated process, where the blend is sent to a fluid catalytic cracking (FCC) unit. The same numbers in FIG. 4 that correspond to FIGS. 2 and 3 refer to the same items/units. As shown, the blend is prepared 25 and then mixed with co-feed Vacuum Gas Oil (VGO) 20. The blend is generally heated to a temperature above the melting point of the plastic before mixing with the co-feed VGO. This co-feed mixture 26 of the blend and conventional VGO refinery feed is then sent to a FCC unit 28 in a refinery. In another embodiment, the heated blend and the VGO co-feed are each passed directly, but separately, to the FCC unit.

The fluid catalytic cracking (FCC) process is widely used in the refining industry for conversion of atmospheric gas oil, vacuum gas oil, atmospheric residues and heavy stocks recovered from other refinery operations into high-octane gasoline, light fuel oil, heavy fuel oil, olefin-rich light gas (LPG) and coke. FCC generally uses a high activity zeolite catalyst to crack the heavy hydrocarbon molecules at a 950-990° F. reactor temperature in a riser with a short contact time of a few minutes or less. LPG streams containing olefins (propylene, butylene) are commonly upgraded to make alkylate gasoline, or to be used in chemicals manufacturing. A conventional FCC unit can be used. However, in the present process for preparing polystyrene, it has been found that by selecting metal oxide additive containing catalysts for the FCC unit, styrene in more significant amounts can be obtained. In one embodiment, the selected catalysts compare a calcium and/or magnesium oxide additive containing zeolite catalyst.

Polystyrene can be produced using free radical polymerization at a temperature of around 120° C. A free radical initiator, such as dibenzoyl peroxide, is used.

The refinery will generally have its own hydrocarbon feed flowing through the refinery units. In this case, as shown in FIG. 4, the hydrocarbon feed is VGO 20. The flow volume of blend to the refinery units, here an FCC unit, can comprise any practical or accommodating volume % of the total flow to the refinery units. Generally, the flow of the blend, for practical reasons, can be up to about 50 vol. % of the total flow, i.e., the refinery flow and the blend flow. In one embodiment, the flow of the blend is an amount up to about 100 vol. % of the total flow. In another embodiment, the volume flow of the blend is an amount up to about 25 vol. % of the total flow. About 50 vol. % has been found to be an amount that is quite practical in its impact on the refinery while also providing excellent results and being an amount that can be accommodated. Avoiding any negative impact on the refinery and its products is important. If the amount of the plastic in the final blend (comprising the plastic/oil blend and co-feed hydrocarbon) is greater than 20 wt. % of the final blend, difficulties in FCC unit operation might ensue. By the final blend is meant the present plastic/oil blend and any co-feed hydrocarbon. The plastic/oil blend can comprise up to 100 vol. % of the feed to the refinery units.

In FIG. 4, cracking of the plastic/hydrocarbon hot blend combined with the co-feed petroleum feed in the FCC unit 28, which comprises a metal oxide additive containing catalyst, produces various olefin/paraffin streams 32, and a gasoline 33 and heavy fraction 30. A stream 31 of aromatics including styrene will also be produced, and the styrene can be separated in a separation unit 37 and passed via 38 to styrene polymerization 40, from which polystyrene products can be made 41.

The C₄ 32 and other hydrocarbon product streams, such as the heavy fraction 30 from the FCC unit 28, are sent to appropriate refinery units 34 for upgrading into clean gasoline, diesel, or jet fuel. The gasoline 33 from the FCC unit may be passed directly to a gasoline pool 35 or further upgraded before sending to a gasoline pool (not shown in the figure).

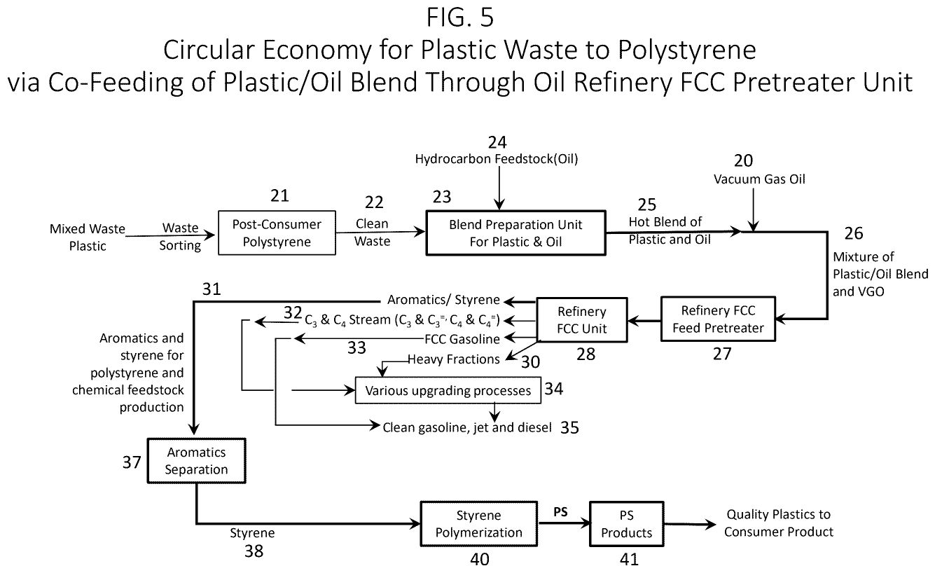

FIG. 5 shows a present integrated process such as that shown in FIG. 4, where the co-feed of the blend and hydrocarbon refinery flow 26 is sent first to a fluid catalytic cracking (FCC) feed pretreater unit 27. The numerals in FIG. 5 which are the same as in FIG. 4 refer to the same streams or refinery units.

The FCC Feed Pretreater typically uses a bimetallic (NiMo or CoMo) alumina catalyst in a fixed bed reactor to hydrogenate the feed with H₂ gas flow at a 660-780° F. reactor temperature and 1,000-2,000 psi pressure. The refinery FCC Feed Pretreater Unit is effective in removing sulfur, nitrogen, phosphorus, silica, dienes and metals that will hurt the FCC unit catalyst performance. Also, this unit hydrogenates aromatics and improves the liquid yield of the FCC unit.

The pretreated hydrocarbon stream from the feed pretreater unit 27 can be distilled to produce various product streams, but generally the stream from the pretreater is sent directly to FCC unit 28 for further production.

The following examples are provided in order to further illustrate the present process. However, the examples are not meant to be limiting. In the following examples, it is demonstrated that significant amounts of styrene are only recovered when using metal oxide catalyst such as a calcium or magnesium oxide containing additive catalyst. Other oxide catalysts can also be used. When solely zeolite containing highly acidic catalysts are used such as ZSM-5 or USY containing ECAT, the amount of styrene is sparse if any at all. There is some styrene recovered when using ZSM-5, but no styrene with USY containing ECAT. Recovered styrene can be used to make a circular polystyrene, as good as virgin polystyrene. This is shown in FIGS. 4 and 5 above.

### EXAMPLES

### Example 1: Properties of Plastic Samples

Two polystyrene (PS) polymer samples with different molecular weights were used for the blend preparations. Their properties of polystyrene is shown in Table 1.

TABLE 1
Properties of Plastics Used

| | PS | PS |
|---|---|---|
| Form | Pellets | Pellets |
| Melt Index | 2.0-4.0 g/10 min | — |
| | (200° C./5 kg) | |
| Melting Point, ° C. | 270 | — |
| Transition Temp, ° C. | 95 | 123-128, |
| | | softening |
| Density, g/mL at 25° C. | 1.04 | 1.06 |
| Average molecular weight, M_{w} | ~350,000 | ~35,000 |
| Number average molecular weight, Mₙ | ~170,000 | |

TABLE 2
Properties of LCO and aromatic blend for Blend Preparation

| | LCO | Aromatic 100 |
|---|---|---|
| | Hydrocarbon | Hydrocarbon |
| | Feed #1 | Feed #2 |
| Specific Gravity | 0.956 | 0.872 |
| Carbon, wt. % | 90.50 | 89.90 |
| Hydrogen, wt. % | 9.50 | 10.10 |
| H/C Molar Ratio | 1.26 | 1.33 |
| Bromine Number | — | — |
| Total S, ppm | 900 | 0 |
| Total N, ppm | N/A | 0 |
| Ni, ppm | <0.2 | 0 |
| V, ppm | <0.2 | 0 |
| Simdist, ° F. | | |

| | | | |
|---|---|---|---|
| IBP | (0.5%) | 235 | 297 |
| 5 | wt. % | 405 | 325 |
| 10 | wt. % | 441 | 327 |
| 30 | wt. % | 490 | 330 |
| 50 | wt. % | 541 | 336 |
| 70 | wt. % | 607 | 344 |
| 90 | wt. % | 689 | 355 |
| 95 | wt. % | 718 | 362 |
| FBP | (99.5%) | 786 | 376 |

Thermal Gravimetric Analysis (TGA) was conducted with the samples of PS and are compared to VGO as well as other plastics such as PVC, LDPE, HDPE and PP in FIG. 6. PS should be stable up to 550° F. so it can be blended without decomposition. The blending temperature should be less than 550° F., and preferably 500° F. or less.

### Example 2—Preparation of Stable Blends of Plastic

Several blends of plastic were prepared by adding plastic pellets to a solvent for blending.

The following procedure is used. The solvents tested are LCO and an aromatic solvent blend, and their properties are summarized in Table 2 above. At ambient temperature, the solvent was added to a beaker and heated with a heating mantle while stirring with a magnetic stirrer. The temperature was raised gradually to 270-400° F., and then pre-weighed plastic pellets (solids) were slowly added to the hot oil while stirring and heating. Visual observation was used to determine if the PS was soluble. If a homogeneous blend could be formed the stirred solution was then held at the final temperature for 60 additional minutes. Upon cooling to ambient temperature, the blend of the plastic and solvent showed the visual appearance of a waxy solid. Results of the solubility trials are shown in Table 3, and it was determined that LCO and the aromatic solvent blend are suitable to dissolve PS so that it can be delivered to a conversion unit such as a FCC unit.

Surprisingly, polystyrene does not make stable blends with many kinds of hydrocarbon solvents, unlike polyethylene and polypropylene. Blends of polystyrene with other hydrocarbons (vacuum gas oil (VGO), soybean oil (SBO), tallow, palm oil, and n-decane each) were attempted using the procedure above. Upon heating in these hydrocarbons, polystyrene pellets gradually melted in the hydrocarbon solvent. However, instead of forming an homogeneous liquid as in the cases with LCO and aromatic solvents, the plastic melt may not be completely miscible. Upon cooling, the plastic phase agglomerated and formed a large plastic solid piece.

Several mixtures bio feedstock and aromatic solvents were prepared. A 50:50 ratio of palm oil and aromatic solvent, 50:50 mixture of tallow and aromatic solvent, and 50:50 ratio of soybean oil and aromatic solvents were prepared. Plastic/bio feedstock/aromatic solvent blend preparations were attempted with 5 wt. % of low molecular weight polystyrene. Surprisingly, none of these mixed solvents of bio feedstock and aromatic solvent was able to produce a stable polystyrene and hydrocarbon blend. The study showed that only an aromatic based solvent can make a stable blend with polystyrene.

The likely explanation for this is that in order to maintain a stable blend of plastic and hydrocarbon mixture, the hydrocarbon solvent must have a very high degree of aromaticity. Aromatic solvent and LCO with abundant aromatic rings are able to solvate the polystyrene polymer strands and able to suspend them in the hydrocarbon solvent to form a stable blend at the ambient temperature.

TABLE 3
Solubility tests of low molecular weight polystyrene
(LMW PS) and high molecular weight polystyrene
(HMW PS) in a variety of potential FCC feeds.

| Hydrocarbon | 10 wt. % | 10 wt. % | 5 wt. % | 5 wt. % |
|---|---|---|---|---|
| Solvent | LMW-PS | HMW-PS | LMW PS | HMW PS |
| LCO | S | S | S | S |
| Aromatics | S | S | S | S |
| Hydrotreated VGO | U | | | |
| SBO | | | U | |
| Tallow | | | U | U |
| Palm | | | U | |
| n-C10 | | | U | |
| Palm/Arom. 50/50 | | | U | |
| Tallow/Arom 50/50 | | | U | |
| SBO/Arom. 50/50 | | | U | |

S = stable mixture formed with no apparent phase separation
U = unstable, no homogeneous blend formed, phase separation

### Catalytic Testing

To study the impact of processing polystyrene in a FCC unit, laboratory tests of a fluidized catalytic cracking (FCC) process were carried out with stable blends of PS. Two FCC catalysts were used for the study: a ZSM-5 FCC catalyst made of ZSM-5 zeolite (10-membered ring medium pore zeolite) and a USY FCC catalyst made of USY (12-membered ring large pore zeolite). In addition to FCC catalysts, an FCC additive containing calcium oxide was also tested.

The catalytic cracking experiments were carried out in an ACE (advanced cracking evaluation) Model C unit fabricated by Kayser Technology Inc. (Texas, USA). The reactor employed in the ACE unit was a fixed fluidized reactor with 1.6 cm ID. Nitrogen was used as fluidization gas and introduced from both bottom and top. The top fluidization gas was used to carry the feed injected from a calibrated syringe feed pump via a three-way valve. The experiments were carried out at atmospheric pressure and a temperature of 975° F. For each experiment a constant amount of 1.5-gram of feed was injected at the rate of 1.2 gram/min for 75 seconds. The cat/oil ratio was kept at 6. After 75 seconds of feed injection, the catalyst was stripped off by nitrogen for a period of 525 seconds. During the catalytic cracking and stripping process the liquid product was collected in a sample vial attached to a glass receiver, which was located at the end of the reactor exit and was maintained at −15° C. The gaseous products were collected in a closed stainless-steel vessel (12.6 L) prefilled with N₂ at 1 atm. Gaseous products were mixed by an electrical agitator rotating at 60 rpm as soon as feed injection was completed. After stripping, the gas products were further mixed for 10 mins to ensure homogeneity. The final gas products were analyzed using a refinery gas analyzer (RGA). After the completion of stripping process, the in-situ catalyst regeneration was carried out in the presence of air at 1300° F. The regeneration flue gas passed through a catalytic converter packed with CuO pellets (LECO Inc.) to oxidize CO to CO₂. The flue gas was then analyzed by an online IR analyzer located downstream of the catalytic converter. Coke deposited during the cracking process was calculated from the CO₂ concentrations measured by the IR analyzer.

Gaseous products, mainly C₁ through C₇ hydrocarbons, were resolved in an RGA. The RGA is a customized Agilent 7890B GC equipped with three detectors, a flame ionization detector (FID) for hydrocarbons and two thermal conductivity detectors for nitrogen and hydrogen. Gas products were grouped into dry gas (C₂-hydrocarbons and hydrogen), LPG (C₃ and C₄ hydrocarbons). Liquid products were weighed and analyzed in a simulated distillation GC (Agilent 6890) using ASTM D2887 method. The liquid products were cut into gasoline (C₅ −430° F.), LCO (430-650° F., light cycle oil) and HCO (650° F.+, heavy cycle oil). Gasoline (C₅+ hydrocarbons) in the gaseous products were combined with gasoline in the liquid products as total gasoline. Light ends in the liquid products (C₅−) were also subtracted from liquid products and added back to C₃ and C₄ species using some empirical distributions. Material balances were between 98% and 101% for most experiments.

Detailed hydrocarbon analysis (DHA) using Agilent 6890A (Separation Systems Inc., FL) were also performed on the gasoline portion of liquid products for PONA and octanes (RON and MON). DHA analysis on the gasoline portion in gaseous products were not performed. The DHA results, however, still provided valuable information to evaluate catalytic cracking product properties.

### Example 3—FCC Evaluation of LCO with MgO/CaO Catalyst (Base Case)

This example shows catalytic conversion of the LCO feed from Example 2 in the presence of a MgO/CaO FCC additive catalyst at three different reactor temperatures using the lab FCC testing unit described above. The results are summarized in Table 4. These results are the base cases to compare with the polystyrene/LCO co-processing cases in Example 4 (Table 5) to establish the impact of catalytic conversion of polystyrene in a fluidized catalytic cracking unit.

TABLE 4
Variable temperature cracking of LCO over calcium oxide
and magnesium oxide containing additive (Base Case)

| | Example | | |
|---|---|---|---|
| | Example 3-1 | Example 3-2 | Example 3-3 |
| | Base case | Base case | Base case |
| | Feed | | |
| | LCO | LCO | LCO |
| | Catalyst | | |
| | MgO/CaO | MgO/CaO | MgO/CaO |
| | additive | additive | additive |
| Temperature (F.) | 775 | 875 | 975 |
| Cat/Oil, wt/wt | 6 | 6 | 6 |
| Conversion [wt. %]* | 11.42 | 12.96 | 16.00 |
| Yield [wt. %] | | | |
| Coke | 4.67 | 5.94 | 7.40 |
| Dry Gas | 0.06 | 0.18 | 0.57 |
| LPG | 0.47 | 0.63 | 1.12 |
| Propylene | 0.16 | 0.24 | 0.45 |
| C4 Olefins | 0.25 | 0.32 | 0.54 |
| Gasoline | 6.21 | 6.23 | 6.91 |
| (C5 - 430° F.) | | | |
| Light Cycle Oil | 75.03 | 72.43 | 69.26 |
| (430° F.-650° F.) | | | |
| Heavy Cycle Oil | 13.55 | 14.60 | 14.74 |
| (650° F.+) | | | |
| Styrene, wt. % | 0.00 | 0.01 | 0.01 |
| of feed | | | |
| Gasoline Properties | | | |
| n-Paraffins [wt. %] | 3.41 | 2.05 | 3.17 |
| Isoparaffins [wt. %] | 3.96 | 3.06 | 2.94 |
| Naphthenes [wt. %] | 0.45 | 0.18 | 0.15 |
| Olefins [wt. %] | 1.25 | 0.85 | 1.07 |
| Aromatics [wt. %] | 87.20 | 91.14 | 90.19 |
| RON** | 85.40 | 85.18 | 84.61 |
| MON** | 74.64 | 73.71 | 73.39 |
| (RON + MON)/2** | 80.02 | 79.45 | 79.00 |

*Conversion - conversion of 430° F.⁺ fraction to 430° F.⁻.
**Octane number, (R + M)/2, was estimated from detailed hydrocarbon GC of FCC gasoline.

Conversion of LCO in the FCC unit is low, ranging from 11.4 to 16.0% with the reactor temperatures from 750° F. to 950° F., resulting in only 6.2 to 6.9 wt. % of gasoline range product. The yield of styrene is negligible in the product for all three base cases.

### Example 4—FCC Evaluation of Polystyrene/LCO Blend with MgO/CaO Catalyst

A 10/90 wt. % blend of polystyrene and LCO feed was evaluated with the FCC unit using MgO/CaO FCC additive catalyst at three different reactor temperatures and the results are summarized in Table 5. The impact of co-processing of polystyrene with LCO by the FCC process is compared with the corresponding base cases in Table 4.

TABLE 5
Variable temperature cracking of 10/90 wt. % blend of PS/LCO
over calcium oxide and magnesium oxide containing additive.
Impact of polystyrene in the feed and temperature.

| | Example | | |
|---|---|---|---|
| | Example 4-1 | Example 4-2 | Example 4-3 |
| | Invention | Invention | Invention |
| | Feed | | |
| | 10 wt. % | 10 wt. % | 10 wt. % |
| | LMW-PS- | LMW-PS- | LMW-PS- |
| | in-LCO | in-LCO | in-LCO |
| | Catalyst | | |
| | MgO/CaO | MgO/CaO | MgO/CaO |
| | additive | additive | additive |
| Temperature (F.) | 775 | 875 | 975 |
| Cat/Oil, wt/wt | 6 | 6 | 6 |
| Conversion [wt. %]* | 16.45 | 18.01 | 20.84 |
| Polystyrene | 61.72 | 63.46 | 64.40 |
| Conversion, [wt. %]** | | | |
| Yield [wt. %] | | | |
| Coke | 6.68 | 4.41 | 4.21 |
| Dry Gas | 0.07 | 0.17 | 0.62 |
| LPG | 0.44 | 0.63 | 1.09 |
| Propylene | 0.16 | 0.24 | 0.44 |
| C4 Olefins | 0.23 | 0.33 | 0.51 |
| Gasoline | 9.26 | 12.80 | 14.93 |
| (C5 - 430° F.) | | | |
| Light Cycle Oil | 70.06 | 67.90 | 65.25 |
| (430° F.-650° F.) | | | |
| Heavy Cycle Oil | 13.49 | 14.08 | 13.90 |
| (650° F.+) | | | |
| Styrene, wt. % of | 1.35 | 2.83 | 3.15 |
| Total Feed | | | |
| Styrene Monomer | 13.50 | 28.30 | 31.50 |
| Yield, wt. % of PS in | | | |
| Total Feed*** | | | |
| Gasoline Properties | | | |
| n-Paraffins [wt. %] | 2.29 | 1.84 | 2.10 |
| Isoparaffins [wt. %] | 2.77 | 2.37 | 1.65 |
| Olefins [wt. %] | 0.66 | 0.64 | 0.97 |
| Naphthenes [wt. %] | 0.20 | 0.22 | 0.26 |
| Aromatics [wt. %] | 91.68 | 93.12 | 94.06 |
| Increase in Aromatics, | 4.48 | 1.98 | 3.87 |
| Wt. % | | | |
| RON**** | 91.28 | 94.87 | 95.32 |
| MON**** | 79.64 | 82.68 | 82.92 |
| (RON + MON)/2**** | 85.46 | 88.78 | 89.12 |

*Conversion - conversion of 430° F.⁺ fraction to 430° F.⁻
**Polystyrene conversion is calculated by subtracting the contribution from LCO and normalizing to 100% polystyrene feed, assuming conversion and yields blend linearly. For example, at 775° F., the polystyrene conversion is calculated as the conversion on PS\LCO blend which is 16.45 minus 90% of neat LCO conversion which is 11.42 and then divided by 10%.
***Styrene monomer yield is the wt. % yield based on the polystyrene in the total feed.
****Octane number, (R + M)/2, was estimated from detailed hydrocarbon GC of FCC gasoline.

Upon addition of polystyrene in the LCO feed to the FCC unit (Example 3-1 vs 4-1; Example 3-2 vs 4-2 Example 3-3 vs 4-3), the conversion increased by about 5 wt. %. As calculated in Table 5, the polystyrene conversion is more than 60%, e.g., more than 60% of polystyrene has been converted to products that boil below 430° F. such as gasoline, LPG and light gases. Styrene yield based on polystyrene in the total feed ranged from 13.50 to 31.50%. The detailed composition analysis of the gasoline boiling range products showed increases in aromatics content upon addition of polystyrene in the feed. The results indicate that good amount of polystyrene has been converted back to styrene monomer and other high-value aromatic compounds.

### Example 5—FCC Evaluation of Polystyrene/LCO Blend with Zeolite Catalysts

Pure LCO feed was evaluated with the FCC unit using FCC catalysts (USY catalyst and ZSM-5 catalyst) as the base cases and the results are summarized in Table 6. A 10/90 wt. % blend of polystyrene and LCO feed was evaluated with the same catalysts to study the impact of co-processing of polystyrene with LCO and the results were compared with the corresponding base cases in Table 6.

TABLE 6
Cracking of Polystyrene/LCO Blend over ECAT
and ZSM-5, Impacts of Polystyrene in the Feed
and Catalyst (USY and ZSM-5 Catalysts)

| | Example | | | |
|---|---|---|---|---|
| | Example | Example | Example | Example |
| | 5-1 | 5-2 | 5-3 | 5-4 |
| | Base case | Invention | Base case | Invention |
| | Feed | | | |
| | | LCO-10 | | LCO-10 |
| | | wt. % | | wt. % |
| | LCO | LMW-PS | LCO | LMW-PS |
| | Catalyst | | | |
| | ECAT | ECAT | ZSM-5 | ZSM-5 |
| Conversion [wt. %] | 34.14 | 44.45 | 15.91 | 23.68 |
| Polystyrene | NA | Complete** | Base Case | 93.61 |
| Conversion, wt. % | | | | |
| Temperature (F.) | 975 | 975 | 975 | 975 |
| Cat/Oil, wt/wt | 6 | 6 | 6 | 6 |
| Yield [wt. %] | | | | |
| Coke | 8.28 | 8.74 | 1.02 | 1.04 |
| Dry Gas | 2.18 | 2.03 | 1.36 | 1.41 |
| LPG | 6.60 | 5.99 | 5.12 | 4.35 |
| Propylene | 2.21 | 2.17 | 2.53 | 2.19 |
| C4 Olefins | 1.90 | 1.76 | 1.69 | 1.47 |
| Gasoline | 17.07 | 27.69 | 8.41 | 16.89 |
| (C5 - 430° F.) | | | | |
| Light Cycle Oil | 53.63 | 45.39 | 67.99 | 61.57 |
| (430° F.-650° F.) | | | | |
| Heavy Cycle Oil | 12.23 | 10.17 | 16.10 | 14.76 |
| (650° F.+) | | | | |
| Styrene, wt. % of | 0.00 | 0.24 | 0.00 | 1.69 |
| Total Feed | | | | |
| Styrene Monomer | Base case | 2.40 | Base case | 16.9 |
| Yield*, wt. % of PS | | | | |
| in Total Feed*** | | | | |
| Gasoline Properties | | | | |
| n-Paraffins [wt. %] | 1.14 | 0.79 | 3.08 | 2.25 |
| Isoparaffins [wt. %] | 8.97 | 4.24 | 6.59 | 3.35 |
| Olefins [wt. %] | 3.29 | 1.27 | 2.04 | 0.43 |
| Naphthenes [wt. %] | 1.33 | 1.82 | 0.36 | 2.10 |
| Aromatics [wt. %] | 83.73 | 91.81 | 85.17 | 90.89 |
| Increase in | Base case | 8.08 | Base case | 5.72 |
| Aromatics, Wt. % | | | | |
| RON | 89.61 | 102.87 | 83.85 | 101.89 |
| MON | 78.94 | 92.79 | 72.92 | 91.63 |
| (RON + MON)/2 | 84.27 | 97.83 | 78.39 | 96.76 |

*Styrene monomer yield is the wt. % yield based on the polystyrene in the total feed.
**Calculated polystyrene conversion is over 100%, which means all polystyrene in the feed has been completely converted to products boil below 430° F.

Upon addition of polystyrene in the LCO feed to the USY catalyst in the FCC unit (Example 5-1 vs. 5-2), the conversion increased by 10.3 wt. %, which gives complete conversion of polystyrene to products boil below 430° F. Other than the shifts of LCO and gasoline yields, the overall yields (coke, light gas, LPG, HCO) are comparable to the base case. The yield of styrene produced was 2.4 wt. % based on the polystyrene in the feed. The detailed composition analysis of the gasoline boiling range products showed 8.08 wt. % increases in aromatics content upon addition of polystyrene in the feed. These results suggest that polystyrene is fully converted in the FCC unit and produced mostly gasoline boiling range, single-ring aromatic hydrocarbons including styrene which are value-added products.

Upon addition of polystyrene in the LCO feed to the ZSM-5 catalyst in the FCC unit (Example 5-3 vs. 5-4), the conversion increased by 7.7 wt. %, which gives a conversion of 93.6 wt. % for the polystyrene. Other than the shifts of LCO and gasoline yields, the overall yields (coke, light gas, LPG, HCO) are comparable to the base case. The yield of styrene produced was 16.9 wt. % based on the polystyrene in the feed. The detailed composition analysis of the gasoline boiling range products showed 5.72 wt. % increases in aromatics content upon addition of polystyrene in the feed. Like the results on USY, the results on ZSM-5 also suggest that polystyrene is fully converted on ZSM-5 under FCC conditions and produced mostly gasoline boiling range, single-ring aromatic hydrocarbons including styrene which are value-added products.

### Example 6—FCC Evaluation of Polystyrene/Aromatic Solvent Blend with Zeolite Catalysts

Neat aromatic solvent feed was evaluated in the lab FCC unit using FCC catalysts (USY catalyst and ZSM-5 catalyst) as the base cases and the results are summarized in Table 7. A 10/90 wt. % blend of polystyrene and aromatic solvent feed was evaluated with the same catalysts to study the impact of co-processing of polystyrene with aromatic solvent and the results were compared with the corresponding base cases in Table 7.

TABLE 7
Cracking of Polystyrene/Aromatic Solvent Blend
over ECAT and ZSM-5, Impacts of Polystyrene in
the Feed and Catalyst (USY and ZSM-5 Catalysts)

| | Example | | | |
|---|---|---|---|---|
| | Example | Example | Example | Example |
| | 6-1 | 6-2 | 6-3 | 6-4 |
| | Base case | Invention | Base case | Invention |
| | Feed | | | |
| | | Aromatics- | | Aromatics- |
| | Aromatic | 10% | Aromatic | 10% |
| | solvent | LMW-PS | solvent | LMW-PS |
| | Catalyst | | | |
| | ECAT | ECAT | ZSM-5 | ZSM-5 |
| Temperature (F.) | 975 | 975 | 975 | 975 |
| Cat/Oil, wt/wt | 6 | 6 | 6 | 6 |
| Conversion [wt. %] | 93.62 | 96.00 | 96.50 | 97.48 |
| Yield [wt. %] | | | | |
| Coke | 2.75 | 4.80 | 0.32 | 0.85 |
| Dry Gas | 1.30 | 1.28 | 2.10 | 1.57 |
| LPG | 1.88 | 1.52 | 2.31 | 1.68 |
| Propylene | 1.05 | 0.87 | 1.66 | 1.18 |
| C4 Olefins | 0.21 | 0.19 | 0.47 | 0.38 |
| Gasoline | 87.71 | 88.41 | 91.78 | 93.40 |
| (C5 - 430° F.) | | | | |
| Light Cycle Oil | 5.06 | 3.02 | 2.73 | 1.80 |
| (430° F.-650° F.) | | | | |
| Heavy Cycle Oil | 1.32 | 0.98 | 0.77 | 0.71 |
| (650° F.+) | | | | |
| Styrene, wt. % of | 0.00 | 0.39 | 0.00 | 3.00 |
| Total Feed | | | | |
| Styrene Monomer | 0.00 | 3.90 | 0.00 | 30 |
| Yield*, wt. % of PS | | | | |
| in Total Feed*** | | | | |
| Gasoline Properties | | | | |
| n-Paraffins [wt. %] | 1.36 | 0.12 | 0.23 | 0.10 |
| Isoparaffins [wt. %] | 2.54 | 2.33 | 0.80 | 1.49 |
| Olefins [wt. %] | 8.53 | 2.49 | 2.24 | 0.25 |
| Naphthenes [wt. %] | 4.32 | 3.57 | 1.75 | 2.11 |
| Aromatics [wt. %] | 83.23 | 91.42 | 95.10 | 96.02 |
| Increase in | Base case | 8.19 | Base case | 0.92 |
| Aromatics, Wt. % | | | | |
| RON | 101.33 | 110.22 | 110.90 | 112.29 |
| MON | 92.60 | 97.38 | 99.12 | 99.43 |
| (RON + MON)/2 | 96.97 | 103.80 | 105.01 | 105.86 |

*Styrene monomer yield is the wt. % yield based on the polystyrene in the total feed.

Upon addition of polystyrene in the aromatic solvent feed to the USY catalyst in the FCC unit (Example 6-1 vs. 6-2), slight increase in conversion and shifts in the overall yields (coke, light gas, LPG, gasoline, LCO, HCO) are observed compared to the base case which already has very high conversion indicating polystyrene is fully converted in the FCC unit. The yield of styrene produced was 3.9 wt. % based on the polystyrene in the feed. The detailed composition analysis of the gasoline boiling range products showed 8.19 wt. % increases in aromatics content upon addition of polystyrene in the feed.

Upon addition of polystyrene in the aromatic solvent feed to the ZSM-5 catalyst in the FCC unit (Example 6-3 vs. 6-4), slight shifts in the overall yields (coke, light gas, LPG, gasoline, LCO, HCO) are observed compared to the base case which already has near complete conversion indicating polystyrene is also fully converted in the FCC unit. The yield of styrene produced was 30 wt. %. The detailed composition analysis of the gasoline boiling range products showed slight increase of 0.92 wt. % in aromatics content upon addition of polystyrene in the feed. The base case already has a very high aromatic content of 95 wt. %

The foregoing results show that hydrocarbon feedstocks such as LCO and aromatic solvent blend can be successfully used to prepare a blend with polystyrene, which blend can then be converted in a refinery conversion unit. The foregoing conversion results further show that the results can be controlled by appropriate selection of the catalyst used in the conversion reactors to provide more significant amounts of styrene, which can be used to prepare new recycled polystyrene.

As used in this disclosure the word “comprises” or “comprising” is intended as an open-ended transition meaning the inclusion of the named elements, but not necessarily excluding other unnamed elements. The phrase “consists essentially of” or “consisting essentially of” is intended to mean the exclusion of other elements of any essential significance to the composition. The phrase “consisting of” or “consists of” is intended as a transition meaning the exclusion of all but the recited elements except for only minor traces of impurities.

As those skilled in the art will appreciate, numerous modifications and variations of the present invention are possible considering these teachings, and all such are contemplated hereby. For example, in addition to the embodiments described herein, the present invention contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

All of the publications cited in this disclosure are incorporated by reference herein in their entireties for all purposes.

What is claimed is:

1. A process for converting waste polystyrene plastic into recycle for polystyrene polymerization comprising:
(a) selecting waste plastics comprising polystyrene;
(b) preparing a blend of hydrocarbon feedstock and the polystyrene waste plastic with the blend comprising about 20 wt. % or less of the polystyrene waste plastic;
(c) passing the blend at a temperature above the melting point of the polystyrene plastic in the blend to a refinery FCC unit;
(d) recovering an aromatics mixture from the FCC unit;
(e) separating styrene from the aromatics mixture; and
(f) passing the styrene to a polymerization unit to prepare polystyrene.

2. The process of claim 1, wherein the blend of (b) is a hot homogeneous blend of waste polystyrene plastic and hydrocarbon oil.

3. The process of claim 1, wherein the blend of (b) is a stable blend of waste polystyrene plastic and hydrocarbon oil.

4. The process of claim 1, wherein the volume flow of the blend to the refinery FCC unit in (c) comprises up to 100 vol. % of the total hydrocarbon flow to the FCC unit.

5. The process of claim 1, wherein the volume flow of the blend to the refinery FCC unit in (c) comprises up to 50 vol. % of the total hydrocarbon flow to the FCC unit.

6. The process of claim 5, wherein the blend flow comprises up to 25 vol. % of the total flow to the FCC unit.

7. The process of claim 1, wherein the blend of hydrocarbon feedstock and selected waste plastic in (b) is prepared by heating the waste plastic above the melting point of the plastic and mixing with the hydrocarbon feedstock, and then cooling the blend to a temperature below the melting point of the waste plastic.

8. The process of claim 1, wherein the hydrocarbon feedstock in the blend comprises light cycle oil (LCO), heavy cycle oil (HCO), FCC naphtha, gasoline, diesel, toluene, and/or aromatic solvent mixture derived from petroleum.

9. The process of claim 1, wherein the hydrocarbon in the blend comprises light cycle oil (LCO) or an aromatic solvent mixture.

10. The process of claim 1, wherein the FCC unit employs a metal oxide containing additive catalyst.

11. The process of claim 1, wherein the catalyst comprises a calcium oxide or magnesium oxide containing additive catalysts.

12. The process of claim 1, wherein styrene products are prepared from the polymerized styrene.

13. The process of claim 1, wherein the hydrocarbon feedstock comprises from 50 wt. % to 99 wt. % of 1-ring, 2-ring, and 3-ring aromatics.
